(19) United States
(12) Patent Application Publication
(10) Pub. No.: US 2003/0139419 A1
(43) Pub. Date: Jul. 24, 2003

(54) Arylpiperazines and arylpiperidines and their use as metalloproteinase inhibiting agents

(76) Inventors: Bernard Barlaam, Reims (FR); Robert Dowell, Macclesfield (GB); Nicholas John Newcombe, Macclesfield (GB); Howard Tucker, Macclesfield (GB); David Waterson, Macclesfield (GB)

Correspondence Address: Janis K Fraser, Fish & Richardson, 225 Franklin Street, Boston, MA, 02110-2804, US

(21) Appl. No.: 10/204,389

(22) PCT Filed: Feb. 15, 2001
(86) PCT No.: PCT/GB01/00616

(30) Foreign Application Priority Data
Feb. 21, 2000 (EP) 00400469.3

(51) Int. Cl. A61K 31/506; A61K 31/496; A61K 31/495; A61K 31/4545; C 07D 4 3/02; C 07D 4 1/02; C07D 241/04

(52) U.S. Cl. 514/252.11; 514/252.14; 514/253.01; 514/255.02; 544/295; 544/360; 544/357; 544/384; 546/193; 546/216; 514/318; 514/317; 514/316

(57) ABSTRACT

Compounds of formula (I) useful as metalloproteinase inhibitors, especially as inhibitors of MMP 13.

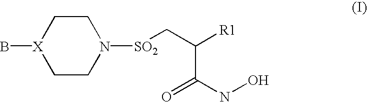

The present invention relates to compounds useful in the inhibition of metalloproteinases and in particular to pharmaceutical compositions comprising these, as well as their use.

The compounds of this invention are inhibitors of one or more metalloproteinase enzymes. Metalloproteinases are a superfamily of proteinases (enzymes) whose numbers in recent years have increased dramatically. Based on structural and functional considerations these enzymes have been classified into families and subfamilies as described in N. M. Hooper (1994) FEBS Letters 354:1-6. Examples of metalloproteinases include the matrix metalloproteinases (MMP) such as the collagenases (MMP 1, MMP8, MMP 13), the gelatinases (MMP2, MMP9), the stromelysins (MMP3, MMP 10, MMP 11), matrilysin (MMP7), metalloelastase (MMP 12), enamelysin (MMP 19), the MT-MMPs (MMP14, MMP15, MMP16, MMP17); the reprolysin or adamalysin or MDC family which includes the secretases and sheddases such as TNF converting enzymes (ADAM10 and TACE); the astacin family which include enzymes such as procollagen processing proteinase (PCP); and other metalloproteinases such as aggrecanase, the endothelin converting enzyme family and the angiotensin converting enzyme family.

Metalloproteinases are believed to be important in a plethora of physiological disease processes that involve tissue remodelling such as embryonic development, bone formation and uterine remodelling during menstruation. This is based on the ability of the metalloproteinases to cleave a broad range of matrix substrates such as collagen, proteoglycan and fibronectin. Metalloproteinases are also believed to be important in the processing, or secretion, of biological important cell mediators, such as tumour necrosis factor (TNF); and the post translational proteolysis processing, or shedding, of biologically important membrane proteins, such as the low affinity IgE receptor CD23 (for a more complete list see N. M. Hooper et al., (1997) Biochem J. 321:265-279).

Metalloproteinases have been associated with many disease conditions. Inhibition of the activity of one or more metalloproteinases may well be of benefit in these disease conditions, for example: various inflammatory and allergic diseases such as, inflammation of the joint (especially rheumatoid arthritis, osteoarthritis and gout), inflammation of the gastro-intestinal tract (especially inflammatory bowel disease, ulcerative colitis and gastritis), inflammation of the skin (especially psoriasis, eczema, dermatitis); in tumour metastasis or invasion; in disease associated with uncontrolled degradation of the extracellular matrix such as osteoarthritis; in bone resorptive disease (such as osteoporosis and Paget's disease); in diseases associated with aberrant angiogenesis; the enhanced collagen remodelling associated with diabetes, periodontal disease (such as gingivitis), corneal ulceration, ulceration of the skin, post-operative conditions (such as colonic anastomosis) and dermal wound healing; demyelinating diseases of the central and peripheral nervous systems (such as multiple sclerosis); Alzheimer's disease; extracellular matrix remodelling observed in cardiovascular diseases such as restenosis and atheroscelerosis; and chronic obstructive pulmonary diseases, COPD (for example, the role of MMPs such as MMP12 is discussed in Anderson & Shinagawa, 1999, Current Opinion in Anti-inflammatory and Immunomodulatory Investigational Drugs, 1(1): 29-38).

A number of metalloproteinase inhibitors are known; different classes of compounds may have different degrees of potency and selectivity for inhibiting various metalloproteinases. We have discovered a new class of compounds that are inhibitors of metalloproteinases and are of particular interest in inhibiting MMP-13, as well as MMP-9. The compounds of this invention have beneficial potency and/or pharmacokinetic properties.

MMP 13, or collagenase 3, was initially cloned from a cDNA library derived from a breast tumour [J. M. P. Freije et al. (1994) Journal of Biological Chemistry 269(24):16766-16773]. PCR-RNA analysis of RNAs from a wide range of tissues indicated that MMP13 expression was limited to breast carcinomas as it was not found in breast fibroadenomas, normal or resting mammary gland, placenta, liver, ovary, uterus, prostate or parotid gland or in breast cancer cell lines (T47-D, MCF-7 and ZR75-1). Subsequent to this observation MMP13 has been detected in transformed epidermal keratinocytes [N. Johansson et al., (1997) Cell Growth Differ. 8(2):243-250], squamous cell carcinomas [N. Johansson et al., (1997) Am. J. Pathol. 151(2):499-508] and epidermal tumours [K. Airola et al., (1997) J. Invest. Dermatol. 109(2):225-231]. These results are suggestive that MMP 13 is secreted by transformed epithelial cells and may be involved in the extracellular matrix degradation and cell-matrix interaction associated with metastasis especially as observed in invasive breast cancer lesions and in malignant epithelia growth in skin carcinogenesis.

Recent published data implies that MMP 13 plays a role in the turnover of other connective tissues. For instance, consistent with MMP 13's substrate specificity and preference for degrading type II collagen [P. G. Mitchell et al., (1996) J. Clin. Invest. 97(3):761-768; V. Knauper et al., (1996) The Biochemical Journal 271:1544-1550], MMP13 has been hypothesised to serve a role during primary ossification and skeletal remodelling [M. Stahle-Backdahl et al., (1997) Lab. Invest. 76(5):717-728; N. Johansson et al., (1997) Dev. Dyn. 208(3):387-397], in destructive joint diseases such as rheumatoid and osteo-arthritis [D. Wernicke et al., (1996) J. Rheumatol. 23:590-595; P. G. Mitchell et al., (1996) J. Clin. Invest. 97(3):761-768; O. Lindy et al., (1997) Arthritis Rheum 40(8):1391-1399]; and during the aseptic loosening of hip replacements [S. Imai et al., (1998) J. Bone Joint Surg. Br. 80(4):701-710]. MMP13 has also been implicated in chronic adult periodontitis as it has been localised to the epithelium of chronically inflamed mucosa human gingival tissue [V. J. Uitto et al., (1998) Am. J. Pathol 152(6):1489-1499] and in remodelling of the collagenous matrix in chronic wounds [M. Vaalarno et al., (1997) J. Invest. Dermatol. 109(1):96-101].

MMP9 (Gelatinase B; 92 kDa TypeIV Collagenase; 92 kDa Gelatinase) is a secreted protein which was first purified, then cloned and sequenced, in 1989 (S. M. Wilhelm et al (1989) J. Biol Chem. 264 (29): 17213-17221. Published erratum in J. Biol Chem. (1990) 265 (36): 22570.). A recent review of MMP9 provides an excellent source for detailed information and references on this protease: T. H. Vu & Z. Werb (1998) (In: Matrix Metalloproteinases. 1998. Edited by W. C. Parks & R. P. Mecham. pp115-148. Academic Press. ISBN 0-12-545090-7). The following points are drawn from that review by T. H. Vu & Z. Werb (1998).

The expression of MMP9 is restricted normally to a few cell types, including trophoblasts, osteoclasts, neutrophils and macrophages. However, it's expression can be induced in these same cells and in other cell types by several mediators, including exposure of the cells to growth factors or cytokines. These are the same mediators often implicated in initiating an inflammatory response. As with other secreted MMPs, MMP9 is released as an inactive Pro-enzyme which is subsequently cleaved to form the enzymatically active enzyme. The proteases required for this activation in vivo are not known. The balance of active MMP9 versus inactive enzyme is further regulated in vivo by interaction with TIMP-1 (Tissue Inhibitor of Metalloproteinases-1), a naturally-occurring protein. TIMP-1 binds to the C-terminal region of MMP9, leading to inhibition of the catalytic domain of MMP9. The balance of induced expression of ProMMP9, cleavage of Pro- to active MMP9 and the presence of TIMP-1 combine to determine the amount of catalytically active MMP9 which is present at a local site. Proteolytically active MMP9 attacks substrates which include gelatin, elastin, and native Type IV and Type V collagens; it has no activity against native Type I collagen, proteoglycans or laminins.

There has been a growing body of data implicating roles for MMP9 in various physiological and pathological processes. Physiological roles include the invasion of embryonic trophoblasts through the uterine epithelium in the early stages of embryonic implantation; some role in the growth and development of bones; and migration of inflammatory cells from the vasculature into tissues. Increased MMP9 expression has observed in certain pathological conditions, thereby implicating MMP9 in disease processed such as arthritis, tumour metastasis, Alzheimer's, Multiple Sclerosis, and plaque rupture in atherosclerosis leading to acute coronary conditions such as Myocardial Infarction.

WO-98/05635 claims compounds of the general formula

B—X—(CH₂)ₙ—CHR¹—(CH₂)ₘ—COY

as having MMP and TNF inhibitory activity.

We have now discovered compounds that are potent MMP13 inhibitors and have desirable activity profiles.

In a first aspect of the invention we now provide compounds of the formula I

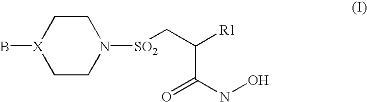

wherein B represents a phenyl group monosubstituted at the 3- or 4-position by halogen or trifluoromethyl, or disubstituted at the 3- and 4-positions by halogen (which may be the same or different); or B represents a 2-pyridyl or 2-pyridyloxy group monosubstituted at the 4-, 5- or 6-position by halogen, trifluoromethyl, cyano or C1-4 alkyl; or B represents a 4-pyrimidinyl group optionally substituted at the 6-position by halogen or C1-4 alkyl;

X represents a carbon or nitrogen atom;

R1 represents a trimethyl-1-hydantoin C2-4alkyl or a trimethyl-3-hydantoin C2-4alkyl group; phenyl or C2-4alkylphenyl monosubstituted at the 3- or 4-position by halogen, trifluoromethyl, thio or C1-3alkyl or C1-3 alkoxy; phenyl-SO2NHC2-4alkyl; 2-pyridyl or 2-pyridyl C2alkyl; 3-pyridyl or 3-pyridyl C2-4alkyl; 2-pyrimidine-SCH2CH2; 2- or 4-pyrimidinyl C1-4alkyl optionally monosubstituted by one of halogen, trifluoromethyl, C1-3 alkyl, C1-3 alkyloxy, 2-pyrazinyl optionally substituted by halogen or 2-pyrazinyl C2-4alkyl optionally substituted by halogen;

Any alkyl groups outlined above may be straight chain or branched.

Preferred compounds of the invention are those wherein any one or more of the following apply:

B represents 4-chlorophenyl, 4-fluorophenyl, 4-bromophenyl or 4-trifluorophenyl; 2-pyridyl or 2-pyridyloxy monosubstituted at the 4- or 5-position such as 5-chloro-⁹-pyridyl, 5-bromo-2-pyridyl, 5-fluoro-2-pyridyl, 5-trifluoromethyl-2-pyridyl, 5-cyano-2-pyridyl, 5-methyl-2-pyridyl; especially 4-fluorophenyl, 5-chloro-2-pyridyl or 5-trifluoromethyl-2-pyridyl;

X represents a nitrogen atom;

R1 is phenylmethyl (or benzyl), phenylethyl (or phenethyl), phenylpropyl, 3-chlorophenyl, 4-chlorophenyl, 3-pyridyl, 2-pyridylpropyl, 2- or 4-pyrimidinylethyl (optionally monosubstituted by fluorine), 2- or 4-pyrimidinylpropyl, 2-(2-pyrimidinyl)propyl (optionally monosubstitued by fluorine); especially phenylmethyl, phenylethyl, 2-pyrimidinylpropyl, 2-(2-pyrimidinyl)propyl (optionally monosubstitued by fluorine) or 5-fluoro-2-pyrimidinylethyl.

For compounds of formula I, a particular subgroup is represented by compounds wherein B is a phenyl group monosubstituted at the 3- or 4-position by halogen or trifluoromethyl, or disubstituted at the 3- and 4-positions by halogen (which may be the same or different); or B is a 2-pyridyl or 2-pyridyloxy group monosubstituted at the 5- or 6-position by halogen, trifluoromethyl or cyano; or B is a 4-pyrimidinyl group optionally substituted at the 6-position by halogen or C1-4 alkyl; X is a carbon or nitrogen atom; R1 is a trimethyl-1-hydantoin C1-4alkyl or a trimethyl-3-hydantoin C2-4alkyl group; or R1 is a phenyl or C1-4alkylphenyl monosubstituted at the 3- or 4-position by halogen, trifluoromethyl, thio or C1-3alkyl or C1-3 alkoxy; or R1 is phenyl-SO2NHC2-4alkyl; or R1 is 2-pyridyl or 2-pyridyl C2-4alkyl; or R1 is 3-pyridyl or 3-pyridyl C2-4alkyl; or R1 is 2-pyrimidine-SCH2CH2; or R1 is 2- or 4-pyrimidinyl C2-4alkyl optionally monosubstituted by one of halogen, trifluoromethyl, C1-3 alkyl, C1-3 alkyloxy, 2-pyrazinyl or 2-pyrazinyl C2-4alkyl; any alkyl group may be straight chain or branched.

It will be appreciated that the particular substituents and number of substituents on B and/or R1 are selected so as to avoid sterically undesirable combinations.

Each exemplified compound represents a particular and independent aspect of the invention.

Where optically active centres exist in the compounds of formula I, we disclose all individual optically active forms and combinations of these as individual specific embodiments of the invention, as well as their corresponding racemates. Racemates may be separated into individual optically active forms using known procedures (cf. Advanced Organic Chemistry: 3rd Edition: author J March, p104-107) including for example the formation of diastereomeric derivatives having convenient optically active auxiliary species followed by separation and then cleavage of the auxiliary species.

It will be appreciated that the compounds according to the invention can contain one or more asymmetrically substituted carbon atoms. The presence of one or more of these asymmetric centres (chiral centres) in a compound of formula I can give rise to stereoisomers, and in each case the invention is to be understood to extend to all such stereoisomers, including enantiomers and diastereomers, and mixtures including racemic is mixtures thereof.

Where tautomers exist in the compounds of formula I, we disclose all individual tautomeric forms and combinations of these as individual specific embodiments of the invention.

As previously outlined the compounds of the invention are metalloproteinase inhibitors, in particular they are inhibitors of MMP13. Each of the above indications for the compounds of the formula I represents an independent and particular embodiment of the invention. Whilst we do not wish to be bound by theoretical considerations, the compounds of the invention are believed to show selective inhibition for any one of the above indications relative to any MMP1 inhibitory activity, by way of non-limiting example they may show 100-1000 fold selectivity over any MMP1 inhibitory activity.

Certain compounds of the invention are of particular use as aggrecanase inhibitors ie. inhibitors of aggrecan degradation. Certain compounds of the invention are of particular use as inhibitors of MMP9 and/or MMP12.

The compounds of the invention may be provided as pharmaceutically acceptable salts. These include acid addition salts such as hydrochloride, hydrobromide, citrate and maleate salts and salts formed with phosphoric and sulphuric acid. In another aspect suitable salts are base salts such as an alkali metal salt for example sodium or potassium, an alkaline earth metal salt for example calcium or magnesium, or organic amine salt for example triethylamine.

They may also be provided as in vivo hydrolysable esters. These are pharmaceutically acceptable esters that hydrolyse in the human body to produce the parent compound. Such esters can be identified by administering, for example intravenously to a test animal, the compound under test and subsequently examining the test animal's body fluids. Suitable in vivo hydrolysable esters for carboxy include methoxymethyl and for hydroxy include formyl and acetyl, especially acetyl.

In order to use a compound of the formula I or a pharmaceutically acceptable salt or in vivo hydrolysable ester thereof for the therapeutic treatment (including prophylactic treatment) of mammals including humans, it is normally formulated in accordance with standard pharmaceutical practice as a pharmaceutical composition.

Therefore in another aspect the present invention provides a pharmaceutical composition which comprises a compound of the formula I or a pharmaceutically acceptable salt or an in vivo hydrolysable ester and pharmaceutically acceptable carrier.

The pharmaceutical compositions of this invention may be administered in standard manner for the disease condition that it is desired to treat, for example by oral, topical, parenteral, buccal, nasal, vaginal or rectal administration or by inhalation. For these purposes the compounds of this invention may be formulated by means known in the art into the form of, for example, tablets, capsules, aqueous or oily solutions, suspensions, emulsions, creams, ointments, gels, nasal sprays, suppositories, finely divided powders or aerosols for inhalation, and for parenteral use (including intravenous, intramuscular or infusion) sterile aqueous or oily solutions or suspensions or sterile emulsions.

In addition to the compounds of the present invention the pharmaceutical composition of this invention may also contain, or be co-administered (simultaneously or sequentially) with, one or more pharmacological agents of value in treating one or more disease conditions referred to hereinabove.

The pharmaceutical compositions of this invention will normally be administered to humans so that, for example, a daily dose of 0.5 to 75 mg/kg body weight (and preferably of 0.5 to 30 mg/kg body weight) is received. This daily dose may be given in divided doses as necessary, the precise amount of the compound received and the route of administration depending on the weight, age and sex of the patient being treated and on the particular disease condition being treated according to principles known in the art.

Typically unit dosage forms will contain about 1 mg to 500 mg of a compound of this invention.

Therefore in a further aspect, the present invention provides a compound of the formula I or a pharmaceutically acceptable salt or in vivo hydrolysable ester thereof for use in a method of therapeutic treatment of the human or animal body. In particular we disclose use in the treatment of a disease or condition mediated by MMP13 and/or aggrecanase and/or MMP9 and/or MMP12.

In yet a further aspect the present invention provides a method of treating a metalloproteinase mediated disease condition which comprises administering to a warm-blooded animal a therapeutically effective amount of a compound of the formula I or a pharmaceutically acceptable salt or in vivo hydrolysable ester thereof. Metalloproteinase mediated disease conditions include arthritis (such as osteoarthritis), atherosclerosis, chronic obstructive pulmonary diseases (COPD).

In another aspect the present invention provides a process for preparing a compound of the formula I or a pharmaceutically acceptable salt or in vivo hydrolysable ester thereof which process comprises conversion of compound II, where Y is a precursor or a protected form of CONHOH. Compound II can be prepared in the following ways

a) by reacting compound III with compound IV, which is obtained conveniently from compound V;

b) by reduction of compound VI, which is conveniently obtained by reacting compound VII with compound VIII;

c) by reaction of compound VII with compound IX where Z is a suitable leaving group.

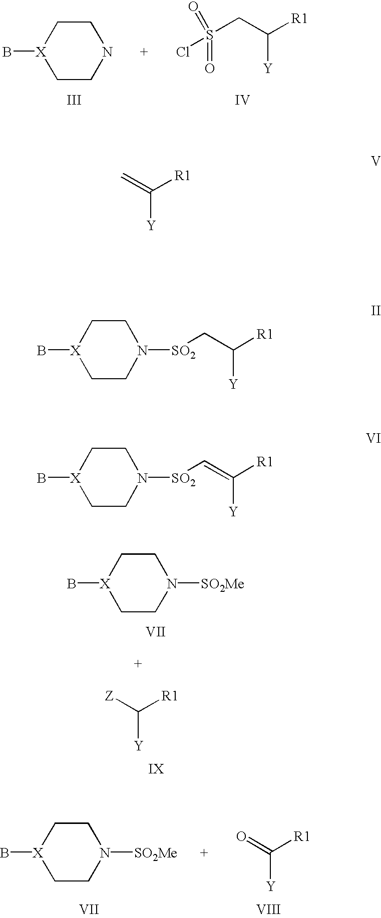

It will be appreciated that many of the relevant starting materials are commercially available or may be found in the scientific literature.

The compounds of the invention may be evaluated for example in the following assays:

Isolated Enzyme Assays

Matrix Metalloproteinase Family Including for Example MMP13

Recombinant human proMMP13 may be expressed and purified as described by Knauper et al. [V. Knauper et al., (1996) The Biochemical Journal 271:1544-1550 (1996)]. The purified enzyme can be used to monitor inhibitors of activity as follows: purified proMMP13 is activated using 1 mM amino phenyl mercuric acid (APMA), 20 hours at 21° C.; the activated MMP13 (11.25ng per assay) is incubated for 4-5 hours at 35° C. in assay buffer (0.1M Tris-HCl, pH 7.5 containing 0.1M NaCl, 20 mM CaCl2, 0.02 mM ZnCl and 0.05% (w/v) Brij 35 using the synthetic substrate 7-methoxycoumarin-4-yl)acetyl.Pro.Leu.Gly.Leu.N-3-(2,4-dinitrophenyl)-L-2,3-diaminopropionyl.Ala.Arg.NH₂ in the presence or absence of inhibitors. Activity is determined by measuring the fluorescence at λex 328 nm and λem 393 nm. Percent inhibition is calculated as follows: % Inhibition is equal to the [Fluorescence_{plus inhibitor}−Fluorescence_{background}] divided by the [Fluorescence_{minus inhibitor}−Fluorescence_{background}].

A similar protocol can be used for other expressed and purified pro MMPs using substrates and buffers conditions optimal for the particular MMP, for instance as described in C. Graham Knight et al., (1992) FEBS Lett. 296(3):263-266.

Adamalysin Family Including for Example TNF Convertase

The ability of the compounds to inhibit proTNFα convertase enzyme may be assessed using a partially purified, isolated enzyme assay, the enzyme being obtained from the membranes of THP-1 as described by K. M. Mohler et al., (1994) Nature 370:218-220. The purified enzyme activity and inhibition thereof is determined by incubating the partially purified enzyme in the presence or absence of test compounds using the substrate 4′,5′-Dimethoxy-fluoresceinyl Ser.Pro.Leu.Ala.Gln.Ala.Val.Arg.Ser.Ser.Ser.Arg.Cys(4(3-succinimid-1-yl)-fluorescein)-NH₂ in assay buffer (50 mM Tris HCl, pH 7.4 containing 0.1% (w/v) Triton X-100 and 2mM CaCl₂), at 26° C. for 18 hours. The amount of inhibition is determined as for MMP13 except λex 490 nm and λem 530 nm were used. The substrate was synthesised as follows. The peptidic part of the substrate was assembled on Fmoc-NH-Rink-MBHA-polystyrene resin either manually or on an automated peptide synthesiser by standard methods involving the use of Fmoc-amino acids and O-benzotriazol-1-yl-N,N,N′,N′-tetramethyluronium hexafluorophosphate (HBTU) as coupling agent with at least a 4- or 5-fold excess of Fmoc-amino acid and HBTU. Ser¹ and Pro² were double-coupled. The following side chain protection strategy was employed; Ser¹(But), Gln⁵(Trityl), Arg^{8,12}(Pmc or Pbf), Ser^{9,10,11}(Trityl), Cys¹³(Trityl). Following assembly, the N-terminal Fmoc-protecting group was removed by treating the Fmoc-peptidyl-resin with in DMF. The amino-peptidyl-resin so obtained was acylated by treatment for 1.5-2 hr at 70° C. with 1.5-2 equivalents of 4′,5′-dimethoxy-fluorescein-4(5)-carboxylic acid [Khanna & Ullman, (1980) Anal Biochem. 108:156-161) which had been preactivated with diisopropylcarbodiimide and 1-hydroxybenzotriazole in DMF]. The dimethoxyfluoresceinyl-peptide was then simultaneously deprotected and cleaved from the resin by treatment with trifluoroacetic acid containing 5% each of water and triethylsilane. The dimethoxyfluoresceinyl-peptide was isolated by evaporation, trituration with diethyl ether and filtration. The isolated peptide was reacted with 4-(N-maleimido)-fluorescein in DMF containing diisopropylethylamine, the product purified by RP-HPLC and finally isolated by freeze-drying from aqueous acetic acid. The product was characterised by MALDI-TOF MS and amino acid analysis.

Natural Substrates

The activity of the compounds of the invention as inhibitors of aggrecan degradation may be assayed using methods for example based on the disclosures of E. C. Arner et al., (1998) Osteoarthritis and Cartilage 6:214-228; (1999) Journal of Biological Chemistry, 274 (10), 6594-6601 and the antibodies described therein. The potency of compounds to act as inhibitors against collagenases can be determined as described by T. Cawston and A. Barrett (1979) Anal. Biochem. 99:340-345.

Inhibition of Metalloproteinase Activity in Cell/tissue Based Activity

Test as an Agent to Inhibit Membrane Sheddases such as TNF Convertase

The ability of the compounds of this invention to inhibit the cellular processing of TNFα production may be assessed in THP-1 cells using an ELISA to detect released TNF essentially as described K. M. Mohler et al., (1994) Nature 370:218-220. In a similar fashion the processing or shedding of other membrane molecules such as those described in N. M. Hooper et al., (1997) Biochem. J. 3921:265-279 may be tested using appropriate cell lines and with suitable antibodies to detect the shed protein.

Test as an Agent to Inhibit Cell Based Invasion

The ability of the compound of this invention to inhibit the migration of cells in an invasion assay may be determined as described in A. Albini et al., (1987) Cancer Research 47:3239-3245.

Test as an Agent to Inhibit Whole Blood TNF Sheddase Activity

The ability of the compounds of this invention to inhibit TNFα production is assessed in a human whole blood assay where LPS is used to stimulate the release of TNFα. Heparinized (10 Units/ml) human blood obtained from volunteers is diluted 1:5 with medium (RPMI1640+bicarbonate, penicillin, streptomycin and glutamine) and incubated (160 μl) with 20μl of test compound (triplicates), in DMSO or appropriate vehicle, for 30 min at 37° C. in a humidified (5%CO₂/95%air) incubator, prior to addition of 20 μl LPS (E. coli. 0111:B4; final concentration 10 μg/ml). Each assay includes controls of diluted blood incubated with medium alone (6 wells/plate) or a known TNFα inhibitor as standard. The plates are then incubated for 6 hours at 37° C. (humidified incubator), centrifuged (2000 rpm for 10 min; 4° C.), plasma harvested (50-100 μl) and stored in 96 well plates at −70° C. before subsequent analysis for TNFα concentration by ELISA.

Test as an Agent to Inhibit in vitro Cartilage Degradation

The ability of the compounds of this invention to inhibit the degradation of the aggrecan or collagen components of cartilage can be assessed essentially as described by K. M. Bottomley et al., (1997) Biochem J. 323:483-488.

Pharmacodynamic Test

To evaluate the clearance properties and bioavailability of the compounds of this invention an ex vivo pharmacodynamic test is employed which utilises the synthetic substrate assays above or alternatively HPLC or Mass spectrometric analysis. This is a generic test which can be used to estimate the clearance rate of compounds across a range of species. Animals (e,g. rats, marmosets) are dosed iv or po with a soluble formulation of compound (such as 20% w/v DMSO, 60% w/v PEG400) and at subsequent time points (e.g. 5, 15, 30, 60, 120, 240, 480, 720, 1220 mins) the blood samples are taken from an appropriate vessel into 10U heparin. Plasma fractions are obtained following centrifugation and the plasma proteins precipitated with acetonitrile (80% w/v final concentration). After 30 mins at −20° C. the plasma proteins are sedimented by centrifugation and the supernatan fraction is evaporated to dryness using a Savant speed vac. The sediment is reconstituted in assay buffer and subsequently analysed for compound content using the synthetic substrate assay. Briefly, a compound concentration-response curve is constructed for the compound undergoing evaluation. Serial dilutions of the reconstituted plasma extracts are assessed for activity and the amount of compound present in the original plasma sample is calculated using the concentration-response curve taking into account the total plasma dilution factor.

In vivo Assessment

Test as an Anti-TNF Agent

The ability of the compounds of this invention as ex vivo TNFα inhibitors is assessed in the rat. Briefly, groups of male Wistar Alderley Park (AP) rats (180-210 g) are dosed with compound (6 rats) or drug vehicle (10 rats) by the appropriate route e.g. peroral (p.o.), intraperitoneal (i.p.), subcutaneous (s.c.). Ninety minutes later rats are sacrificed using a rising concentration of CO₂ and bled out via the posterior vena cavae into 5 Units of sodium heparin/ml blood. Blood samples are immediately placed on ice and centrifuged at 2000 rpm for 10 min at 4° C. and the harvested plasmas frozen at −20° C. for subsequent assay of their effect on TNFα production by LPS-stimulated human blood. The rat plasma samples are thawed and 175 μl of each sample are added to a set format pattern in a 96U well plate. Fifty μl of heparinized human blood is then added to each well, mixed and the plate is incubated for 30 min at 37° C. (humidified incubator). LPS (25 μl; final concentration 10 μg/ml) is added to the wells and incubation continued for a further 5.5 hours. Control wells are incubated with 25μl of medium alone. Plates are then centrifuged for 10 min at 2000 rpm and 200 μl of the supernatans are transferred to a 96 well plate and frozen at −20° C. for subsequent analysis of TNF concentration by ELISA.

Data Analysis by Dedicated Software Calculates for each Compound/dose:

$$Percent inhibition of TNFα = \frac{Mean TNFα ( Controls ) - Mean TNFα ( Treated ) \times 100 }{ M ean TNFα ( Controls )}$$

Test as an Anti-arthritic Agent

Activity of a compound as an anti-arthritic is tested in the collagen-induced arthritis (CIA) as defined by D. E. Trentham et al., (1977) J. Exp. Med. 146,:857. In this model acid soluble native type II collagen causes polyarthritis in rats when administered in Freunds incomplete adjuvant. Similar conditions can be used to induce arthritis in mice and primates.

Test as an Anti-cancer Agent

Activity of a compound as an anti-cancer agent may be assessed essentially as described in I. J. Fidler (1978) Methods in Cancer Research 15:399-439, using for example the B 16 cell line (described in B. Hibner et al., Abstract 283 p75 10th NCI-EORTC Symposium, Amsterdam Jun. 16-19 1998).

The invention will now be illustrated but not limited by the following Examples:

### EXAMPLE 1

### N-hydroxy-3-[4-fluorophenylpiperidin-1-ylsulphonyl]-2-benzylpropionamide

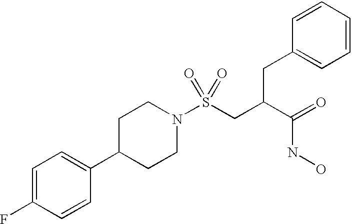

A solution of 3-[4-fluorophenylpiperidin-1-ylsulphonyl]-2-benzyl-N-benzyloxypropionamide (75 mg) in ethanol (2 ml) containing 10% palladium on carbon (8 mg) was hydrogenated under a hydrogen filled balloon. The catalyst was filtered and the solvent removed under vacuum. The residue was passed through a Bond-elute column eluting with a mixture of ethyl acetate and isohexane (1:1) to give the title compound, yield 29 mg as a white foam. M+H=421. ¹H nmr (300 MHz, d⁶-DMSO+d³AcOD) d 1.45-1.65 (m, 2H,); 1.7-1.8 (m, 2H,); 2.5-2.6 (m [partly obscured by solvent], 2H,); 2.65-2.9 (m, 5H,); 3.4-3.5 (m, 1H,); 3.5-3.6 (m, 2H,); 7.1 (dd, 2H,); 7.2-7.3 (m, 7H

### 3-[4-fluorophenylpiperidin-1-ylsulphonyl]-2-benzyl-N-benzyloxypropionamide

A solution of 3-chlorosulphonyl-2-benzyl-N-benzyloxypropionamide (720 mg) in methylene chloride (2 ml) was added dropwise to a solution of 4-fluorophenylpiperidine (320 mg) and triethylamine (306 μl) in methylene chloride (6 ml) at 0° C. The reaction mixture was stirred for 14 hours, washed with water and filtered through phase separating paper and evaporated to dryness. The residue was purified by chromatography through a Bond-elute column with a mixture of ethyl acetate and isohexane (1:4) as eluant to give the title compound as a white solid, yield 75 mg, M+H=511. ¹H nmr (300 MHz, CDCl₃) d 1.65-1.85 (2×m, 4H); 2.45-2.6 (m, 1H,); 2.65-3.1 (m, 6H); 3.6 (dd, 1H); 3.75-3.85 (m, 2H); 4.5 (Abq, 0.5H); 4.65-4.8 (m, 0.5H); 4.8 (Abq, 0.5H,); 4.95-5.1 (m, 0.5H); 6.9-7.0 (m, 2H); 7.1-7.15 (m, 2H); 7.15-7.2 (m, 2H); 7.3-7.4 (m, 8H)

### 3-Chlorosulphonyl-2-benzyl-N-benzyloxypropionamide

Chlorine was passed into a vigorously stirred mixture of 3-acetylthio-2-benzyl-N-benzyloxypropionamide (750 mg) in methylene chloride (5 ml) and water (5 ml) at 10° C. Chlorine flow was stopped when the reaction mixture became yellow and stirring was continued for 14 hours. The reaction mixture was purged with argon and extracted with methylene chloride (3×10 ml). The combined extracts were dried and solvent removed to give the title compound as a yellow oil, yield 725 mg. This was used without further characterization.

### 3-Acetylthio-2-benzyl-N-benzyloxypropionamide

A mixture of N-benzyloxy-2-benzylacrylamide (0.61 g) and thiolacetic acid (0.32 ml) was stirred and heated at 70° C. for 3 hours. Toluene (5 ml) was added to the reaction mixture which was evaporated to dryness to give the title compound as a gum (M+H=344) which was used without further characterization.

### N-Benzyloxy-2-benzylacrylamide

One drop of DMF was added to a mixture of 2-benzylacrylic acid (0.4g) (CAS No 5669-19-2) and oxalyl chloride (0.22 ml) in methylene chloride (5 ml) and the mixture was stirred for 30 minutes. The solvent was removed and methylene chloride (5 ml) was added and this, in turn, was removed. The residue was dissolved in methylene chloride (2 ml) and this was added to a solution of O-benzylhydroxylamine hydrochloride (0.39 g) and triethylamine (0.69 ml) in methylene chloride. The mixture was stirred for 1 hour, washed with water (2×10 ml) and dried. The residue obtained on removal of the solvent was passed down a Bond-elute column eluting with methylene chloride initially but then in a gradient with ethyl acetate (up to 10% ethyl acetate/methylene chloride) to give the title compound, yield 420 mg as a gum, M+H=268. 1H-NMR (CDCl₃): 3.6 (s, 2H), 4.83 (s, 2H), 5.25 (s, 1H), 5.58 (s, 1H), 7.1-7.37 (m, 10H), 8.1 (s, 1H).

### EXAMPLE 2

### N-hydroxy-3-[4-fluorophenylpiperazin-1-ylsulphonyl]-2-benzylpropionamide

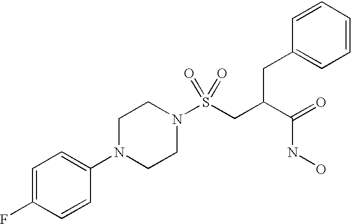

A solution of 3-[4-fluorophenylpiperazin-1-ylsulphonyl]-2-benzyl-N-benzyloxypropionamide (234 mg) in methanol containing 10% palladium on carbon (30 mg) was hydrogenated under a hydrogen filled balloon for 3.5 hours. The catalyst was removed by filtration through Celite and the filtrate was evaporated to dryness to give the title compound, yield 165 mg, M+H=422. ¹H-NMR (CDCl₃): 2.8-3.6 (m, 14H), 6.8 (dd, 2H), 6.9 (t, 2H), 7.4-7.9 (m, 5H).

### 3-[4-fluorophenylpiperazin-1-ylsulphonyl]-2-benzyl-N-benzyloxypropionamide

A mixture of 3-[N-(4-fluorophenyl)piperazin-1-ylsulphonyl]-2-benzylpropionic acid. (203 mg), carbon tetrabromide (182 mg), triethylamine (0.209 ml), O-benzylhydroxylamine (76 mg) and polymer supported triphenylphosphine (500 mg) in methylene chloride (5 ml) was stirred for 14 hours. The reaction mixture was diluted with methylene chloride (10 ml) and aminomethylated polystyrene (1 g) was added and the mixture was stirred for 4 hours, filtered through silica (2 g) washing with methylene chloride. The filtrate was evaporated to dryness and the residue was purified by chromatography on silica eluting with increasing volumes of ethyl acetate in isohexane (5% initially increasing to 50%). The title compound was obtained as a clear gum, 237 mg, M−H=510. ¹H-NMR (CDCl₃): 2.75 (b, 1H), 2.95 (m, 3H), 3.1 (b, 4H), 3.35 (b, 4H), 3.6 (m, 1H), 4.6 (d. 1H), 4.8 (d, 1H), 6.85 (q, 2H), 6.95 (t, 2H), 7.15-7.35 (m, 10H), 8.0 (b, 1H).

### 3-[N-(4-fluorophenyl)piperazin-1-ylsulphonyl]-2-benzylpropionic Acid

Lithium hydroxide (14 ml of a 1 M aqueous solution) was added to a solution of ethyl 3-[N-(4-fluorophenyl)piperazin-1-ylsulphonyl]-2-benzylpropionate (1 g) in THF (20 ml) and stirred vigorously for 4 hours. The reaction mixture was acidified to pH 1 with hydrochloric acid (10 ml of 1.5M) and extracted with ethyl acetate (3×25 ml). The ethyl acetate extracts were washed with water and dried. The residue obtained on evaporated to dryness was triturated with diethyl ether to give the title compound as a white solid, yield 219 mg, ¹H-NMR (CDCl₃): 2.9 (dd, 1H), 3.0 (dd, 1H), 3.1 (t, 1H), 3.15 (dd, 1H), 3.25 (m, 1H), 3.35 (m, 2H), 3.45 (dd, 1H), 6.85 (dd, 2H), 6.95 (t, 2H), 7.2-7.25 (m, 5H).

### Ethyl 3-[N-(4-fluorophenyl)piperazin-1-ylsulphonyl]-2-benzylpropionate

A mixture of N-(4-fluorophenyl)-piperazine (9.01 g) and triethylamine (7.0 ml) in methylene chloride (150 ml) was added dropwise to a cooled (−15° C.) solution of 2-ethoxycarbonyl-3-phenylpropanesulphonyl chloride (15.0 g) in methylene chloride (75 ml) at such a rate that the internal temperature did not exceed −5° C. The mixture was stirred for 15 minutes and quenched with dilute HCl (15 ml of 1.5M), washed with water (2×100 ml) and brine (50 ml)>. The aqueous extracts were washed with methylene chloride (100 ml) and the combined organic extracts were dried. The residue obtained on removal of the solvent was purified by chromatography on silica eluting with a mixture of ethyl acetate and isohexane (1:5) to give the title compound, yield 12.02 g, M+H=435 (434). ¹H-NMR (CDCl₃): 1.2 (t, 3H), 2.85-3.0 (b, 2H), 3.0-3.2 (b, 5H), 3.25 (b, 1H), 3.35 (b, 2H), 3.45 (dd, 1H), 4.15 (q, 2H), 6.85 (b, 2H), 7.0 (b, 2H), 7.15-7.4 (m, 5H).

### 2-Ethoxycarbonyl-3-phenylpropanesulphonyl Chloride

Chlorine gas was bubbled into a suspension of ethyl-2-(acetylthiomethyl)-3-phenylpropionate (16 g) until the reaction mixture became yellow. The reaction mixture was purged with nitrogen and the mixture was concentrated under reduced pressure. The residue was extracted with methylene chloride (2×200 ml) washed with brine (50 ml) and dried to give the title compound as a yellow oil, yield 15.0 g which was used without further purification. ¹H-NMR (CDCl₃): 1.2 (t, 3H), 2.95 (dd, 1H), 3.2 (dd, 1H), 3.45 (q, 1H), 3.65 (dd, 1H), 4.2 (m, 3H), 7.1-7.4 (m, 5H).

### Ethyl-2-(acetylthiomethyl)-3-phenylpropionate

A mixture of ethyl 2-benzylacrylate (CAS No. 20593-63-9) (20 g) and thiolacetic acid (14.2 g) was heated at 70° C. for 14 hours. The mixture was concentrated under reduced pressure and the residue was passed through silica (50 g) eluting with an ethyl acetate/isohexane mixture (1:9) to give the title compound as a yellow oil, yield 31 g. ¹H-NMR (CDCl₃): 1.15 (t, 3H), 2.3 (s, 3H), 2.8-3.2 (m, 5H), 4.1 (q, 2H), 7.1-7.3 (m, 5H).

### EXAMPLE 3

### [(4-fluorophenyl)-4-(piperazinylsulphonyl)]-2-N-hydroxycarboxamide-4-phenylbutane

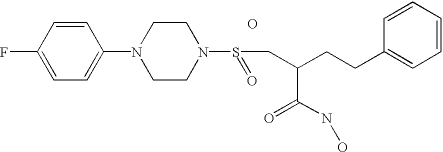

[(4-fluorophenyl)-4-(piperazinylsulphonyl)]-2-carboxylic acid-4-phenylbutane (490 mg) was suspended in dichloromethane (5 mL), cooled to 5° C. and DMF (2 μL) added followed by oxalyl chloride (0.43 mL) at such a rate to keep the temperature at 5-7° C. After 1 hr at this temperature the mixture was evaporated to dryness and azeotroped with toluene to give a yellow oil. This oil was dissolved in dichloromethane (5 mL) and added to a cooled solution of 50% aqueous hydroxylamine (0.3 mL) in THF (10 mL) at 5° C. After 10 mins. the mixture was evaporated to dryness and partitioned between ethyl acetate and water. The organic phase was dried and evaporate to dryness. Trituration with ether gave [(4-fluorophenyl)-4-piperazinylsulphonyl)]-2-N-hydroxycarboxamide-4-phenylbutane as a solid (300 mg).

NMR CDCl₃ d 7.3-6.8, (m, 9H); 3.5 (m 1H); 3.1, (m, 4H); 3.3, (m, 4H); 2.8-2.5 (m, 4H); 1.9-.2.2. (br, 2H);

Mass spec. MH+ 436

### [(4-fluorophenyl)-4-(piperazinylsulphonyl)]-2-carboxylic acid-4-phenylbutane

[4-fluorophenyl)-4-(piperazinlsulphonyl)]-2-ethoxycarbonyl-4-phenylbutane (1.7 g) was dissolved in a mixture of THF (25 mL) and water (8 mL) and lithium hydroxide monohydrate (190 mg) was added. The mixture was stirred at ambient temperature for 18 hrs. and then evaporated to almost dryness. 1.0M lithium hydroxide solution (200 mL) was added and the solution extracted with ether (100 mL). The aqueous phase was acidified to pH 4 with citric acid and extracted with ethyl acetate. The extracts were dried and evaporated to give [(4-fluorophenyl)-4-(piperazinylsulphonyl)]-2-carboxylic acid-4-phenylbutane (540 mg).

NMR DMSO d 7.3-6.8, (m, 9H); 3.6 (m 1H); 3.5, (m, 1H); 3.4, (m, 4H); 3.15, (m, 4H); 2.8 (m, 2H); 2.7, (m, 2H); 1.9-.2.2. (br, 2H);

Mass spec. MH+ 421

### [(4-fluorophenyl)-4-(piperazinylsulphonyl)]-2-ethoxycarbonyl-4-phenylbutane

E-[(4fluorophenyl)-4-(piperazinylsulphonyl)]-2-ethoxycarbonyl-4-phenylbut-1-ene (10 g, 0.022M) was dissolved in tetrahydrofuran (50 mL) and ethanol (500 mL) at 30-35° C. Sodium borohydride (2.09 g. 0.055M) was added, in portions, keeping temperature below 35° C. The mixture was stirred for 15 minutes water (100 mL) was added and the pH adjusted to 4 with 1M citric acid solution. The mixture was evaporated to dryness and the residue partitioned between dichloromethane and water. The combined organic phases were dried and evaporated to dryness. The residue was purified by flash column chromatography eluting with iso-hexane/ethyl acetate 3:1 to yield [(4-fluorophenyl)-4-(piperazinylsulphonyl)]-2-ethoxycarbonyl-4-phenylbutane a white solid.(1.9 g)

NMR d 7.3-6.8, (m, 9H); 4.2, (m, 2H); 3.5, (m, 1H); 3.4, (m, 4H); 3.15, (m, 4H); 3.0, (m, 2H); 2.7, (m, 2H); 2.2-2.1, (br, 2H); 1.3, (t,3H).

MS MH+ 449.

### E-[(4-fluorophenyl)-4-(piperazinylsulphonyl)]-2-ethoxycarbonyl-4-phenylbut-1-ene

N-(4-fluorophenyl)-N′-(methanesulphonyl) piperazine (12.9 g, 0.05M) was dissolved in dry tetrahydrofuran (500 mL) and cooled to −10° C. under an argon atmosphere. A 1.0M solution of lithium bis(trimethylsilyl)amide in tetrahydrofuran (100 mL, 0.1M) was added dropwise at −10° C., stirred for 30 minutes, then added chlorotrimethyl silane (5.45 g, 6.36 ml, 0.05M)keeping the temperature at −10° C. After stirring at −10° C. for a further 30 minutes a solution of ethyl-2-oxo-phenylbutyrate (10.3 g, 9.5 ml, 0.05M)in tetrahydrofuran (20 ml) was added dropwise. After stirring at −10° C. for 1 hour the reaction was quenched with saturated ammonium chloride solution. Diluted with ethyl acetate, collected the organic phase, dried and evaporated to dryness. The residual oil, which was a mixture of E and Z isomers, was separated by column chromatography on silica gell eluting with iso-hexane/ethyl acetate 3:1 to yield E-[(4-fluorophenyl)-4-(piperazinylsulphonyl)]-2-ethoxycarbonyl-4-phenylbut-1-ene as the less polar isomer (6.6 g)

NMR d 7.3-6.6, (m, 9H); 5.8, (s 1H); 4.2, (m, 2H); 3.0 (m, 4H); 2.9, (m, 4H); 2.7, (m,2H); 2.55, (m, 2H); 1.15, (t, 3H)

MS MH+ 447, M+Na 469, MH− 445

### N-(4-fluorophenyl)-N′-(methanesulphonyl)piperazine

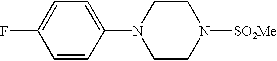

To a solution of 1-(4-fluorophenyl)piperazine (35 g, 194 mmol) and pyridine (17.5 ml) in dry dichloromethane (200 ml) at 0° C. was added methanesulfonyl chloride (20 ml, 258 mmol) dropwise. The mixture was stirred for 3 hours at room temperature. The mixture was washed with water and extracted with dichloromethane (2×100 ml). The organic layers were dried with MgSO₄ and evaporated in vacuo. The residue was triturated and washed with methanol to give 1-(4-fluorophenyl)-4-(methanesulfonyl)piperazine (39.35 g) as white crystals.

¹H NMR (CDCl₃): 7.00 (m, 2H), 6.90 (m, 2H), 3.40 (m, 4H), 3.20 (m, 4H), 2.83 (s, 3H).

### EXAMPLE 4

### 3-{[4-(5-chloropyrid-2-yl)piperazino]sulfonyl}-N-hydroxy-2-phenylpropanamide

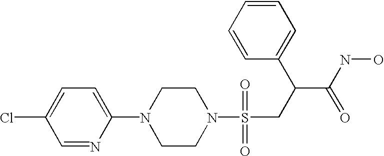

A solution of 3-{[4-(5-chloropyrid-2-yl)piperazino]sulfonyl}-2-phenylpropanoic acid (416mg, 1.02 mmol) in DCM (3.5 ml) with DMF (1 drop) was stirred at 0° C., under an Argon blanket. Oxalyl chloride (0.266 ml, 3.05 mmol) was added dropwise and the reaction was stirred for 30 mins. The mixture was evaporated in vacuo and azeotroped with toluene. The resultant yellow oil was taken into DCM (2.5 ml) and added dropwise to a solution of hydroxyl amine (50% aqueous solution, 0.333 ml) in THF (2.5 ml) at 0° C. Stirred for 30 mins at 5° C. before evaporating in vacuo to a gum. The residue was taken into EtOAc before washing with water (×2), then dried over Na₂SO₄ and evaporated in vacuo to afford pale yellow foam (0.250 g). ¹H NMR (DMSO): 10.85 (s, 1H), 8.92 (s, 1H), 8.10 (d, 1H), 7.62 (dd, 1H), 7.40-7.18 (m, 5H), 6.90 (d, 1H), 4.40-3.80 (m, 2H), 3.56 (m. 3H), 3.48 (m, 1H), 3.25 (m, 1H), 3.20 (m, 3H); MS (ES+): 425.2 (MH⁺).

The starting material was prepared as follows:

2-(N-methanesulfonylpiperazine)-5-chloropyridine (1.0 g, 3.63 mmol) was taken into anhydrous THF (50 ml) under Argon then cooled to −10° C. before the addition of Li(TMSA) (3.8 ml of a 1.0M solution in THF, 3.81 mmol). The mixture was stirred at −10° C. for 10 minutes before dropwise addition of a pre-prepared solution [→-bromophenylacetic acid (1.24 g, 5.81 mmol) treated with Li(TMSA) (6.1 ml of a 1.0M solution in THF, 6.10 mmol) in THF (40 ml) at −10° C., under Argon]. The suspension mixture was stirred at −10° C. for 30 mins then allowed to warm to RT. Quenched with aqueous ammonium chloride and acidified with conc. HCl to pH2 before extracted with ethyl acetate (×3). The organic layers were dried over Na₂SO₄ and evaporated in vacuo to afford a yellow gum. The gum was dissolved in a small amount of EtOAc and precipitated with Et₂O. Filtered and washed with Et₂O to afford a white solid (0.522 g). ¹H NMR (DMSO): 7.95 (d, 1H), 7.45 (dd, 1H), 7.22-7.08 (m, 5H), 6.75 (d, 1H) 3.82-3.74 (m, 2H,), 3.38 (m, 4H), 3.23 (m, 1H), 3.04 (m, 4H), 2.50 (m, 1H); MS (ES+): 410.4 (MH⁺).

### 2-(N-methanesulfonylpiperazine)-5-chloropyridine

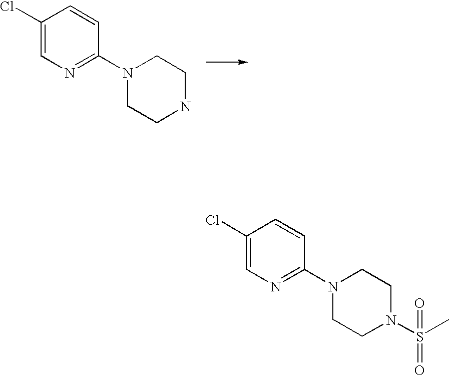

5-Chloro-2-piperazinopyridine (95.1 g, 0.48M) was dissolved in CH2Cl2 (1000 ml and triethylamine (67.6 ml, 0.48M) was added. Cooled to 0-5 C. and slowly added a solution of methane sulphonyl chloride (37.4 ml, 0.48M)in CH2Cl2 (50 ml). The reaction mixture was stirred at room temperature overnight. Washed the reaction mixture with H2O (300 ml). Collected the organic phase, dried over MgSO4, filtered and evaporated to dryness to yield a white solid. The solid was stirred in ethanol (500 ml) at 60 C. Cooled and collected the white solid. Dried at 40 C. under vacuum overnight. Yield 97.3 g.

NMR (CDCl₃) d 8.1, d 1H; 7.4, dd 1H; 6.6, d 1H; 3.7, m 4H; 3.3, m 4H; 2.8, s 3H.

MS Found MH+ 276

### 5-Chloro-2-piperazinopyridine

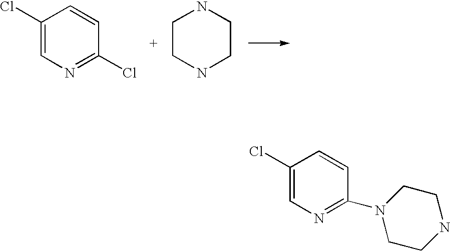

2,5-Dichloropyridine (148 g, 1.0M) was dissolved in anhydrous dimethylacetamide (1000 ml) and anhydrous piperazine (258 g, 3.0M) was added. Stirred at 120 C. for 4 hours. Cooled and evaporated under hi-vac on cold-finger buchi. The residue was stirred in ethyl acetate (3000 ml). Filtered of the solid, washing with ethyl acetate (500 ml) The combined ethyl acetate filtrates were washed with H2O, dried over MgSO4, filtered and evaporated to yield a yellow solid. Yield 1 82.5 g.

NMR (CDCl₃) d 8.1, d 1H; 7.4 dd 1H; 6.6, d 1H; 3.5, m 4H; 3.0, m 1H;

MS found MH+ 198

### EXAMPLE 5

### (R,S)-N-Hydroxy-3-[4-fluorophenylpiperazin-1-ylsulphonyl]-2-[(R,S)-2-phenylpropyl]propionamide

The compound was prepared using the method given in Example 1. Below are listed the intermediates and final product.

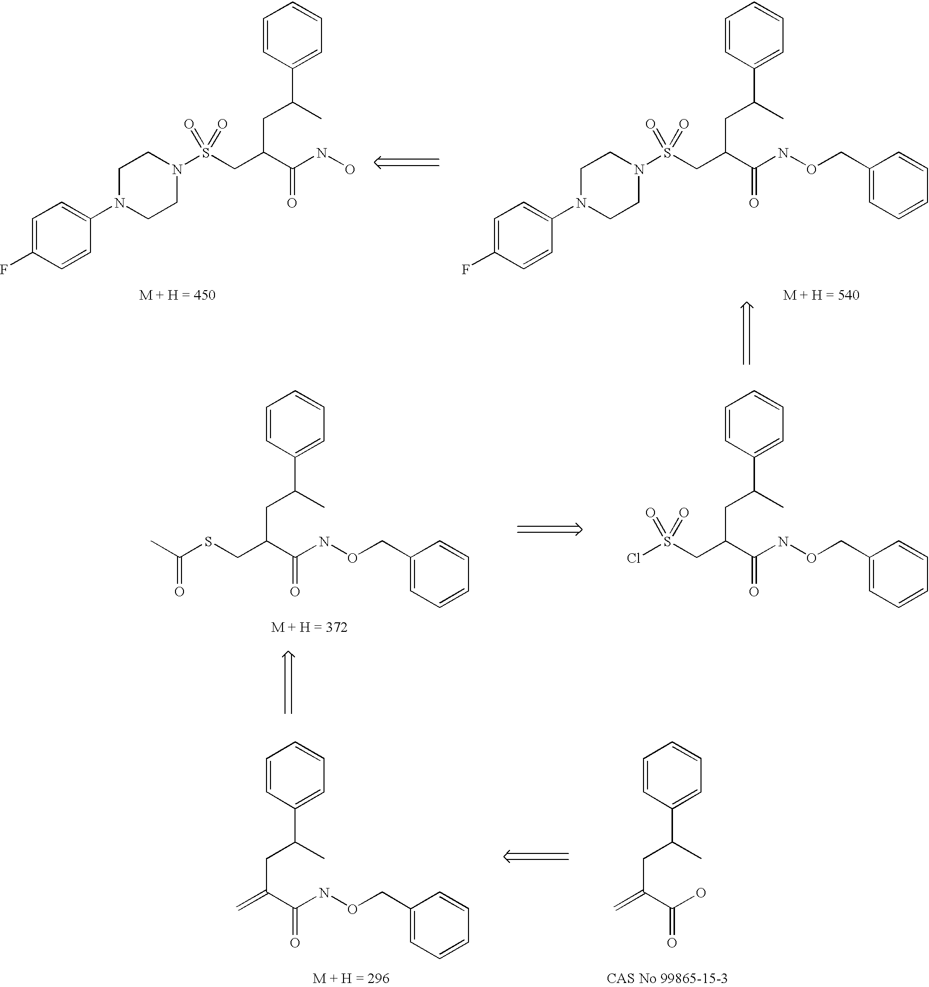

### EXAMPLE 6

The following compounds were prepared using the method given in Example 4.

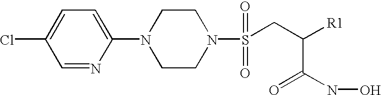

| R1 | M + H |
|---|---|
| 4-Cl—PhCH2 | 473/475 |
| Ph(CH2)2 | 453/455 |
| 4-Cl—Ph | 459/461 |
| 3,4-Dichloro-Ph | 493/495 |
| 2-Pyrimidinyl(CH2)3 | 469 |

### EXAMPLE 7

The following compound was prepared using the method given in Example 4.

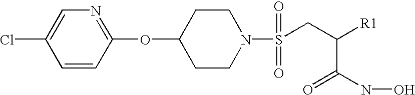

| R1 | M + H |
|---|---|
| Ph(CH2)2 | 468/470 |

What we claim is:

1. A compound of the formula I or a pharmaceutically acceptable salt or an in vivo hydrolysable ester thereof 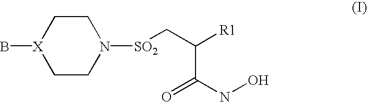
wherein
B is a phenyl group monosubstituted at the 3- or 4-position by halogen or trifluoromethyl, or disubstituted at the 3- and 4-positions by halogen (which may be the same or different); or B is a 2-pyridyl or 2-pyridyloxy group monosubstituted at the 4-, 5- or 6-position by halogen, trifluoromethyl, cyano or C1-4 alkyl; or B is a 4-pyrimidinyl group optionally substituted at the 6-position by halogen or C1-4 alkyl;
X is a carbon or nitrogen atom;
R1 is a trimethyl-1-hydantoin C2-4alkyl or a trimethyl-3-hydantoin C2-4alkyl group; or R1 is phenyl or C2-4alkylphenyl monosubstituted at the 3- or 4-position by halogen, trifluoromethyl, thio or C1-3alkyl or C1-3 alkoxy; or R1 is phenyl-SO2NHC2-4alkyl; or R1 is 2-pyridyl or 2-pyridyl C2-4alkyl; or R1 is 3-pyridyl or 3-pyridyl C2-4alkyl; or R1 is 2-pyrimidine-SCH2CH2; or R1 is 2- or 4-pyrimidinyl C2-4alkyl optionally monosubstituted by one of halogen, trifluoromethyl, C1-3 alkyl, C1-3 alkyloxy, 2-pyrazinyl optionally substituted by halogen or 2-pyrazinyl C2-4alkyl optionally substituted by halogen.

2. A compound as claimed in claim 1 or a pharmaceutically acceptable salt or an in vivo hydrolysable ester thereof wherein:
B is a phenyl group monosubstituted at the 3- or 4-position by halogen or trifluoromethyl, or disubstituted at the 3- and 4-positions by halogen (which may be the same or different); or B is a 2-pyridyl or 2-pyridyloxy group monosubstituted at the 5- or 6-position by halogen, trifluoromethyl or cyano; or B is a 4-pyrimidinyl group optionally substituted at the 6-position by halogen or C1-4 alkyl;
X is a carbon or nitrogen atom;
R1 is a trimethyl-1-hydantoin C2-4alkyl or a timethyl-3-hydantoin C2-4alkyl group; or R1 is phenyl or C2-4alkylphenyl monosubstituted at the 3- or 4-position by halogen, trifluoromethyl, thio or C1-3alkyl or C1-3 alkoxy; or R1 is phenyl-SO2NHC2-4alkyl; or R1 is 2-pyridyl or 2-pyridyl C2-4alkyl; or R1 is 3-pyridyl or 3-pyridyl C2-4alkyl; or R1 is 2-pyrimidine-SCH2CH2; or R1 is 2- or 4-pyrimidinyl C2-4alkyl optionally monosubstituted by one of halogen, trifluoromethyl, C1-3 alkyl, C1-3 alkyloxy, 2-pyrazinyl or 2-pyrazinyl C2-4alkyl.

3. A compound as claimed in claim 1 or a pharmaceutically acceptable salt or an in vivo hydrolysable ester thereof wherein B is selected from 4-chlorophenyl, 4-fluorophenyl, 4-bromophenyl, 4-trifluorophenyl, 5-chloro-2-pyridyl, 5-bromo-2-pyridyl, 5-fluoro-2-pyridyl, 5-trifluoromethyl-2-pyridyl, 5-cyano-2-pyridyl, 5-methyl-2-pyridyl.

4. A compound as claimed in claim 3 or a pharmaceutically acceptable salt or an in vivo hydrolysable ester thereof wherein B is 4-fluorophenyl, 5-chloro-2-pyridyl or 5-trifluoromethyl-2-pyridyl.

5. A compound as claimed in any one of the previous claims or a pharmaceutically acceptable salt or an in vivo hydrolysable ester thereof wherein X is a nitrogen atom.

6. A compound as claimed in any one of the previous claims or a pharmaceutically acceptable salt or an in vivo hydrolysable ester thereof wherein R1 is selected from phenylmethyl, phenylethyl, phenylpropyl, 3-chlorophenyl, 4-chlorophenyl, 3-pyridyl, 2-pyridylpropyl, 2- or 4-pyrimidinylethyl (optionally monosubstituted by fluorine), 2- or 4-pyrimidinylpropyl, 2-(2-pyrimidinyl)propyl (optionally monosubstitued by fluorine).

7. A compound as claimed in claim 6 or a pharmaceutically acceptable salt or an in vivo hydrolysable ester thereof wherein R1 is phenylmethyl, phenylethyl, 2-pyrimidinylpropyl, 2-(2-pyrimidinyl)propyl (optionally monosubstitued by fluorine) or 5-fluoro-2-pyrimidinylethyl.

8. A compound as claimed in claim 1 or a pharmaceutically acceptable salt or an in vivo hydrolysable ester thereof wherein the compound of the formula I is as exemplified herein.

9. A compound as claimed in claim 8 or a pharmaceutically acceptable salt or an in vivo hydrolysable ester thereof wherein the compound is selected from (R,S)-N-Hydroxy-3-[4-fluorophenylpiperazin-1-ylsulphonyl]-2-[(R,S)-2-phenylpropyl]propionamide, 3-{[4-(5-chloropyrid-2-yl)piperazino]sulfonyl}-N-hydroxy-2-phenylpropanamide, [(4-fluorophenyl)-4-(piperazinylsulphonyl)]-2-N-hydroxycarboxamide-4-phenylbutane, N-hydroxy-3-[4-fluorophenylpiperazin-1-ylsulphonyl]-2-benzylpropionamide, N-hydroxy-3-[4-fluorophenylpiperidin-1-ylsulphonyl]-2-benzylpropionamide.

10. A pharmaceutical composition which comprises a compound of the formula I as claimed in claim 1 or a pharmaceutically acceptable salt or an in vivo hydrolysable ester thereof and a pharmaceutically acceptable carrier.

11. A compound of the formula I as claimed in claim 1 or a pharmaceutically acceptable salt or in vivo hydrolysable ester thereof for use in a method of therapeutic treatment of the human or animal body.

12. A compound of the formula I as claimed in claim 1 or a pharmaceutically acceptable salt or in vivo hydrolysable ester thereof for use as a therapeutic agent.

13. A method of treating a metalloproteinase mediated disease condition which comprises administering to a warm-blooded animal a therapeutically effective amount of a compound of the formula I or a pharmaceutically acceptable salt or in vivo hydrolysable ester thereof.

14. A method of treating a metalloproteinase mediated disease condition as claimed in claim 13 which comprises treating a disease condition mediated by one or more of the following enzymes: MMP13, aggrecanase, MMP9, MMP12.

15. The use of a compound of the formula I or a pharmaceutically acceptable salt or in vivo hydrolysable precursor thereof in the preparation of a medicament for use in the treatment of a disease condition mediated by one or more metalloproteinase enzymes.

16. The use of a compound of the formula I or a pharmaceutically acceptable salt or in vivo hydrolysable precursor thereof in the preparation of a medicament for use in the treatment of arthritis.

17. The use of a compound of the formula I or a pharmaceutically acceptable salt or in vivo hydrolysable precursor thereof in the preparation of a medicament for use in the treatment of atherosclerosis.

18. The use of a compound of the formula I or a pharmaceutically acceptable salt or in vivo hydrolysable precursor thereof in the preparation of a medicament for use in the treatment of chronic obstructive pulmonary diseases.

19. A process for preparing a compound of the formula I or a pharmaceutically acceptable salt or in vivo hydrolysable ester thereof which process comprises converting a compound of the formula II to a compound of the formula I 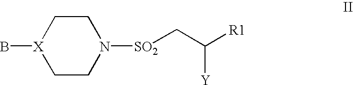
wherein Y is a precursor or a protected form of CONHOH, and optionally thereafter forming a pharmaceutically acceptable salt or in vivo hydrolysable ester of the compound of formula I.
